(19) United States
(12) Patent Application Publication
(10) Pub. No.: US 2007/0025977 A1
(43) Pub. Date: Feb. 1, 2007

(54) METHOD OF TREATING STEATORRHEA IN INFANTS

(76) Inventor: Andrew E. Mulberg, Cherry Hill, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/457,912

(22) Filed: Jul. 17, 2006

Related U.S. Application Data
(60) Provisional application No. 60/701,453, filed on Jul. 21, 2005.

(51) Int. Cl. A61K 38/46

(52) U.S. Cl. 424/94.6

(57) ABSTRACT

A method for treating steatorrhea in infants in need thereof by administering to the infant an amount of pancrelipase of from about 300 to about 2,500 USP units lipase/kg/meal or from about 1,500 to about 7,500 USP units lipase/kg/day.

### CROSS-REFERENCE TO RELATED APPLICATIONS

This application claims the benefit of U.S. Provisional Application 60/701,453, filed on Jul. 21, 2005, which is incorporated by reference herein in its entirety.

### FIELD OF THE INVENTION

This invention is directed to the treatment of cystic fibrosis-(CF) related pancreatice insufficiency (PI) and fat malabsorption. More particularly, this invention is directed towards treating infants and toddlers with CF.

### BACKGROUND OF THE INVENTION

CF is the most common autosomal recessive genetic disorder caused by a mutation in the CF transmembrane conductance regulator (CFTR) gene affecting Caucasians. CF is characterized by a secretory defect that is seen in all epithelial cells resulting from an abnormal gene product, encoding for an abnormal protein called the CFTR. The prevalence of CF in the United States (US) is 1 in 3000 with more than 30,000 patients in the US and 40,000 worldwide affected by the disorder. There are more than 1000 mutations that currently encode for an abnormal CFTR, 70% of which are comprised by the ΔF508 mutation in Caucasians.

Durie and colleagues previously demonstrated that the condition of fat malabsorption (steatorrhea) is related to pancreatic insufficiency (PI), which causes the patient with CF to have severe fat and nutrient malabsorption. There is a strong correlation between the genetic mutation in CF and pancreatic sufficiency (PS) or PI. For example, there is a greater than 90% prevalence of PI in patients with homozygous ΔF508 mutation.

The majority of patients with CF who express the ΔF508 and other genotypes have PI, which manifests as malabsorption. PI is seen in infants and is associated with a more severe genotype. PI can start in utero, and can be associated with meconium ileus in 10% to 15% of infants with PI. Optimal management of the malabsorption seen in those patients with PI results in improved nutritional status which is often associated with fat-soluble vitamin deficiencies, edema, hypoproteinemia, weight loss, and failure to thrive. PS is seen in up to 15% of patients and is associated with recurrent pancreatitis, less severe disease, and longer survival. Some PS patients go on to develop PI; monitoring of pancreatic function is important as infants and children age; requirements for supplementation of pancreatic enzyme therapy characteristically changes with age as nutritional intake varies.

PI is recognized as a clinically important contributor to malnutrition and poor growth in children, e.g., infants and toddlers, with CF. The potential for insufficient and ineffective treatment with supplemental pancreatic enzymes continues to be a clinical problem. Konstan has demonstrated that growth indices and signs and symptoms of lung disease in early childhood are independently associated with reduced lung function at 6 years of age, suggesting that both early nutritional intervention and early treatment of lung disease may improve lung health. Aggressive nutritional rehabilitation and addressing the sources of nutritional deprivation may ultimately affect survival.

More than 15% of children with CF are below the 5th percentile for weight (16.2%) and height (14.6%). The mechanism of poor growth in infants and children may be related to sub-optimal treatment of PI, decreased intake and anorexia, central nervous system dysregulation including increased metabolism and needs not met by intake. Both European and US consensus conferences provided recommendations for the management of the malabsorptive state. For example, while no specific product is mentioned, 1000-2500 USP lipase units/kg/meal, (depending on fat intake for each patient) is recommended. Higher doses can increase the risk of fibrosing colonopathy.

A novel formulation of pancreatic enzyme therapy is pancrelipase microtablets which were introduced by Johnson & Johnson in 1988. Pancrelipase microtablets, derived from porcine pancreatic extracts and given orally, are released from enteric-coated capsules into the lumen of the GI tract. Pancreatic enzymes are a life-saving and vital therapy in the treatment of children and adults with PI, including patients with CF. Pancreatic enzymes are not absorbed and exert their action locally. Capsules contain enteric-coated 2 mm microtablets of porcine pancreatic enzyme concentrate, predominantly lipase, amylase and protease. Pancrelipase microtablets 4, 10 and 16 capsules utilize identical regular potency microtablets and differ only in fill weight of the capsules. The primary indication for pancrelipase microtablets is for the treatment of steatorrhea (fat malabsorption) secondary to PI in disorders, such as CF and chronic pancreatitis.

Current recommendations based on clinical practice guidelines for utilization of pancreatic enzymes indicate that they should be administered to all CF infants who are fed infant formula and eating solid foods since they contain macronutrients that can be malabsorbed. The current recommendation for pancrelipase treatment is to open the capsule(s) and place the beads on a spoon containing a small amount of applesauce, infant rice cereal, banana, or sweet potato baby food. The baby should be given this enzyme-food mixture before the liquid feeding. The mouth should be checked to make sure that all the beads have been swallowed. If retained in the mouth, the beads may irritate the mucous membranes. Published data, though, contradict these recommendations demonstrating the lack of stability of nonenteric-coated lipase activity in applesauce and stability in and in foods containing close to neutral pH. Supplementation in infants, should take into consideration the potential for mouth injury secondary to the failure of swallowing of the entire dose. There is a notable lack of efficacy and safety data in infants and young children less than 2 years of age, which necessitates further investigation into the pharmacodynamic and efficacy aspects of pancreatic enzyme supplementation in this vulnerable cohort of patients.

Colonic strictures, particularly in children with CF, have been associated with doses generally above the recommended dosing range. Patients currently receiving doses >2,500 USP lipase units/kg/meal, or 4,000 USP lipase units/gm fat/day, should be re-evaluated and the dosage either immediately decreased or titrated downward to the lowest effective clinical dose as assessed by 3-day fecal fat excretion.

Dosage should be individualized and determined by the degree of steatorrhea and the fat content of the diet. Therapy should be initiated at the lowest possible dose and gradually increased until the desired control of steatorrhea is obtained. Dosage should be adjusted based on 3-day fecal fat studies.

Current dosing for PANCREASE® MT capsules states that there is considerable variation among individuals in response to enzymes with respect to control of steatorrhea; therefore, a range of doses is suggested. For infants (up to 12 months), a fat-consumption scheme is used where 2,000-4,000 USP lipase units per 120 mL of formula, or per breast feeding, is recommended. This provides approximately 450-900 USP lipase units/g/fat ingested (based on 4.5 grams of fat per 120 mL standard cow's milk-based infant formula). Higher doses are used in infants because, on average, infants ingest 5 grams of fat per kilogram of body weight per day, whereas adults tend to ingest about 2 grams of fat per kilogram per day.

For older children, a weight-based scheme is recommended where for children less than about 4 years, it is recommended to begin with 1,000 USP lipase units/kg/meal to a maximum of 2,500 USP lipase units/kg/meal. For children older than 4 years, it is recommended to begin with 400 USP lipase units/kg/meal to a maximum of 2,500 USP lipase units/kg/meal.

Enzyme doses, expressed as USP lipase units/kg/meal, should be decreased in older patients since they weigh more but tend to ingest less fat per kilogram. Usually, half the mealtime dose is given with a snack. The total daily dose reflects approximately three meals and two to three snacks per day.

If doses greater than 2,500 USP lipase units/kg/meal (4,000 USP lipase units/gm fat/day) are required to control malabsorption, further investigation is warranted to rule out other causes of malabsorption. Doses greater than 2,500 USP lipase units/kg/meal should be used with caution and only if they are documented to be effective by 3-day fecal fat measures. It is unknown whether doses above 2,500 USP lipase units/kg/meal are safe.

Colonic strictures, particularly in children with CF, have been associated with doses generally above the recommended dosing range. Patients currently receiving doses >2,500 USP lipase units/kg/meal or 4,000 USP lipase units/gm fat/day should be re-evaluated and the dosage either immediately decreased or titrated downward to the lowest effective clinical dose as assessed by 3-day fecal fat excretion.

Given the issues associated with large doses of lipase, a dosing schedule for infants that is based on weight, rather than estimated fat consumption, is needed. The present invention surprisingly demonstrated efficacious doses for treating steatorrhea in infants with CF at much lower lipase doses than current recommendation.

### SUMMARY

The present invention provides a method for treating steatorrhea in an infant in need thereof comprising, consisting of, and/or consisting essentially of administering to the infant an amount of pancrelipase of from about 300 to about 2,500 USP units lipase/kg/meal.

The present invention also provides a method for treating steatorrhea in an infant in need thereof comprising, consisting of, and/or consisting essentially of administering to the infant an amount of pancrelipase of from about 1,500 to about 7,500 USP units lipase/kg/day.

### DETAILED DESCRIPTION

As used herein, the term “infant” means a human child between about 6 and about 30 months of age.

Capsules are a pancreatic enzyme supplement for oral administration. Pancrelipase, the active ingredient in PANCREASE® MT capsules, is a natural product harvested by extraction from the pancreas of the hog. Pancrelipase powder is a slightly brown amorphous powder with a faint characteristic odor. It is partly soluble in water and practically insoluble in alcohol or ether. PANCREASE® MT capsules contain enteric-coated microtablets of porcine pancreatic enzyme concentrate in the following theoretical quantities:

PANCREASE ® MT 4 Capsules:
Lipase 4,000 U.S.P. Units
Amylase 12,000 U.S.P. Units
Protease 12,000 U.S.P. Units
PANCREASE ® MT 10 Capsules:
Lipase 10,000 U.S.P. Units
Amylase 30,000 U.S.P. Units
Protease 30,000 U.S.P. Units
PANCREASE ® MT 16 Capsules:
Lipase 16,000 U.S.P. Units
Amylase 48,000 U.S.P. Units
Protease 48,000 U.S.P. Units
PANCREASE ® MT 20 Capsules:
Lipase 20,000 U.S.P. Units
Amylase 56,000 U.S.P. Units
Protease 44,000 U.S.P. Units

The enteric-coated microtablets contained in PANCREASE® MT capsules resist gastric inactivation and deliver enzymes into the duodenum. The enzymes in PANCREASE® MT act locally in the gastrointestinal tract. The enzymes are present in the form of pH-sensitive entericcoated microtablets of 2 mm diameter which are filled into gelatin capsules. The microtablets, which are released from the capsule into the stomach, are enteric-coated to resist inactivation at low pH. Once released, the microtablets are distributed into the stomach and pass into the duodenum where, when the pH reaches approximately 5.5, the enteric coating begins to dissolve and release of the enzymes is initiated. The enzymes catalyze the hydrolysis of fats into glycerol and fatty acids, protein into proteoses and derived substances, and starch into dextrins and sugars. Duodenal availability studies in adults indicate that following oral administration of PANCREASE® MT to adults, measurable levels of enzymes are present in the duodenum. Once they have accomplished their digestive function, the enzymes may be digested in the intestine. The constituents may be partially absorbed and subsequently excreted in the urine. Any undigested enzymes are excreted in the feces.

The inactive ingredients in PANCREASE® MT capsules are cellulose, crospovidone, magnesium stearate, colloidal silicon dioxide, methacrylic acid copolymer, triethyl citrate, talc, polydimethylsiloxane, wax, gelatin, iron oxide, polysorbate 80, sodium lauryl sulfate, titanium dioxide, and other trace ingredient.

PANCREASE® MT is indicated for the treatment of steatorrhea secondary to PI such as CF or chronic alcoholic pancreatitis. PANCREASE® MT capsules are contraindicated in patients known to be hypersensitive to pork protein or any other component of this product.

Any pancrelipase oral enteric coated capsule product is useful in this invention. For example, other branded pancrease lipase oral enteric coated capsules that are useful in the present invention include Cotazym-S (Organon US Inc.), Creon (Solvay), Pancron (Pecos Pharmaceutical Inc.), and Ultrase (Axcan pharma, Inc.).

The invention illustratively disclosed herein suitably may be practiced in the absence of any component, ingredient, or step which is not specifically disclosed herein. Several examples are set forth below to further illustrate the nature of the invention and the manner of carrying it out. However, the invention should not be considered as being limited to the details thereof.

### EXAMPLE 1

In order to develop a stronger insight into the pharmacodynamic effects of intra-luminal pancreatic enzymes in infants and children with CF, this study will include a quantifiable measure of lipase activity intra-luminally using the stable isotope 13C-mixed triglyceride assay. The opportunity of developing further understanding of the pharmacodynamic effects of intraluminal active pancreatic enzymes on lipid digestion will be easily acquired through this non-invasive technique.

This study will provide novel and potentially useful data to assess the safety, palatability, and efficacy of four doses of pancrelipase microtablets in the treatment of infants and toddlers with CF-related PI with fat malabsorption.

The 13C mixed triglyceride (MTG) breath test demonstrates duodenal lipolysis due to both residual endogenous and exogenous pancrease activity. For CF patients, both European and US consensus guidelines provide recommendations for pancreatic enzyme replacement therapy. While no specific product is mentioned, 1000-2000 U lipase/kg/meal is recommended. Nevertheless, these doses have not been studied and there is no published literature on safe and effective doses of enzyme therapy in young infants and children less than 4 years of age. Aims: To assess the efficacy of four dose levels of pancrelipase microtablets (Pancrease® MT) in the treatment of infants with CF-related PI with fat malabsorption.

Methods: Eighteen infants, 6-30 months of age, were provided 500 USP units lipase/kg/meal of pancrelipase microtablets (Pancrease® MT) for five days (120 hours) on an outpatient basis (baseline period). A 13C MTG breath test was performed on each infant after ingesting a liquid or solid test meal containing 13C MTG. Breath samples were collected every 15 min for 6 hrs. The percentage of exhaled 13CO2 was measured using Isotope Ratio Mass Spectrometry (IRMS) and the cumulative expired 13C was calculated. Subsequently, the infants were randomly assigned to one of four treatment groups in a 1:1:1:1 ratio as follows: 500 USP units lipase/kg/meal, 1000 USP units lipase/kg/meal, 1500 USP units lipase/kg/meal, or 2000 USP units lipase/kg/meal for five days (120 hours) on an outpatient basis (randomization period). A second breath test was performed using the same methods.

Results: Of the 18 subjects, 3 dropped out and 3 provided inadequate breath samples for analysis due to low CO2 content. The 12 paired breath tests had been randomized 3 per dosage group. The study group included 8 girls and 4 boys aged 17.09+/−8.29 mo. Mean expired cumulative 13CO2 after the first breath test, with a dosage of 500 USP units lipase/kg/meal, in the 12 subjects was 15.32+/−18.09%. The mean percent difference between the first and second breath test was −1.77% for the 500 USP units lipase/kg/meal, −1.6% for the 1000 USP units lipase/kg/meal, 15.35% for the 1500 USP units lipase/kg/meal, and 125.32% for the 2000 USP units lipase/kg/meal subjects respectively. The mean expired cumulative 13CO2 for the 2000 USP units lipase/kg/meal subjects was 17.60+/_{—}27.12%.

Previous dosing prior to screening averaged over 8500 USP units lipase/kg/day for the infants. During the study the four treatment groups averaged 1833, 4479, 6312, and 7316 USP units lipase per/kg/day, respectively. 12/16 subjects were stable on a lower dose of enzymes during the study. 50% of subjects were managed with less than ½ maintenance enzyme dose.

Conclusions: PANCREASE® MT is clinically effective in the treatment of steatorrhea in infant pediatric subjects ages 6-30 months with CF. The 13C MTG breath test demonstrated pancreatic enzyme activity, i.e., effectiveness, in the low normal range after 500 USP units lipase/kg/meal, which is below the current recommendations for the management of steatorrhea (based on US and European Union treatment guidelines. Increasing the dosage to 1500 and 2000 USP units lipase/kg/meal improved lipolysis as demonstrated in the infants. Mean values are subject to great variability due to a wide range of underlying residual endogenous lipase activity. There were no significant safety issues.

What is claimed:

1. A method for treating steatorrhea in an infant in need thereof comprising administering to the infant an amount of pancrelipase of from about 300 to about 2,500 USP units lipase/kg/meal.

2. The method of claim 1, wherein the amount of pancrelipase is from about 500 to about 2,000 USP units lipase/kg/meal.

3. A method for treating steatorrhea in an infant in need thereof comprising administering to the infant an amount of pancrelipase of from about 1,500 to about 7,500 USP units lipase/kg/day.
